(19) United States
(12) Patent Application Publication
(10) Pub. No.: US 2023/0001105 A1
(43) Pub. Date: Jan. 5, 2023

(54) DEVICE AND METHOD FOR STANDARDIZING SITE ASSESSMENT OF CATHETER INSERTION SITE

(71) Applicant: Becton, Dickinson and Company, Franklin Lakes,, NJ (US)

(72) Inventors: Ashley Rachel Rothenberg, Morris Plains, NJ (US); Erik Kurt Witt, Oakland, NJ (US); Anant Subramaniam, kearny, NJ (US); S. Ray Isaacson, Layton, UT (US); Vincent J. Sullivan, Cary, NC (US); Kenneth G. Powell, Raleigh, NC (US); Charles D. Shermer, Raleigh, NC (US); Brendan Tompkins, Cary, NC (US)

(21) Appl. No.: 17/778,977

(22) PCT Filed: Nov. 20, 2020
(86) PCT No.: PCT/US20/61476

§ 371 Date: May 23, 2022

Related U.S. Application Data
(60) Provisional application No. 62/939,275, filed on Nov. 22, 2019.

(51) Int. Cl. A61M 5/42

(52) U.S. Cl. CPC A61M 5/427; A61M 2205/3306; A61M 2205/50; A61M 2025/0266

(57) ABSTRACT

A method for site assessments of a catheter insertion site and/or dressing includes: scanning the catheter insertion site and/or dressing with an image capture device and/or sensor; selecting a patient baseline site location and skin tone; recording a baseline condition using a computing device; determining a site assessment rate using a computing device; prompting a clinician to make a site assessment of the catheter insertion site and/or dressing using a computing device; and recording site assessment information in an electronic medical record using a computing device.

### CROSS-REFERENCE TO RELATED APPLICATIONS

This application is the United States national phase of International Application No. PCT/US2020/061476 filed Nov. 20, 2020, and claims priority to U.S. Provisional Application Ser. No. 62/939,275, filed Nov. 22, 2019, entitled “Device and Method for Standardizing Site Assessment of Catheter Insertion Site”, the entire disclosures of which are hereby incorporated by reference in their entirety.

### BACKGROUND OF THE DISCLOSURE

The present application relates generally to a system and method for site assessments of catheter insertion site and/or dressing.

### DESCRIPTION OF RELATED ART

Catheter site assessments are routine observations performed by nurses (or physicians, technicians, and other clinicians) on patients that have an intravenous catheter (peripheral or central). The nurse inspects the catheter insertion site to confirm: the catheter is patent and functioning; the catheter is properly in place and has not been dislodged; the dressing is secure, is not peeling, dirty or other compromised; and the skin around the catheter insertion site is not discolored, warm, or soft.

Site assessments are instrumental in detecting if there are any catheter-related complications (including infiltrations, extravasations, phlebitis, dislodgement, etc.) or local site infections. These assessments are expected to be performed at a fixed frequency, depending on the type of catheter, infusate, and patient-specific risk factors. For example, infusing vancomycin (a venous irritant) through a peripheral IV catheter (PIVC) requires a nurse to assess the catheter site every 2 hours to ensure there is no infiltration. The site assessments can be performed as a standalone observation or as part of another procedure (medication delivery, dressing change, overall patient assessments etc.). The site assessments are documented in the electronic medical record (EMR).

Site assessments typically involve the following: inspecting the IV catheter insertion site for redness, swelling, or bruising (the patient's other extremity may be utilized for comparison); assessing for tenderness and swelling through palpating the skin around the catheter site; assessing the condition of the transparent catheter dressing, including if it is clean, dry, and securely adhered to the skin around the insertion site; assessing if any additional securement is used to keep the dressing intact (e.g. additional tape, bandages etc.); assessing the catheter for patency, typically by flushing the catheter with saline or heparin; and assessing products used to secure and stabilize the catheter (particularly for central lines). In the EMR, the nurse will also document the reasons for continuing to use the catheter, and assess for signs of infiltration, phlebitis, dislodgement, and migration.

There are several challenges with site assessments, including: site assessments are inherently subjective, and depends heavily on the observer's awareness and experience; a less experienced floor nurse may observe a site to appear normal while a trained nurse may spot early signs of infiltration or phlebitis; patient-specific factors (e.g. skin color, skin folds, natural discoloration) can confound results; variability in products used to secure and stabilize catheters, leading to variable catheter-related outcomes; little data or evidence to help nurses identify what is an acceptable versus unacceptable catheter site (e.g. how much dressing peeling is acceptable, how much local redness is acceptable); due to documentation overload, nurses may copy and paste assessments, without even observing it on the patient; high frequency site assessments create a workflow and documentation burden for the nurse, which increases likelihood of resorting to short-cuts; and site-assessments are not tracked or validated for accuracy since the only data available is that documented by the nurse in the EMR. There is also an opportunity to evaluate if site assessments can be used as predictors of an impending catheter-related complication, including comparison of a site over time to enable earlier detecting of complications.

### SUMMARY OF THE DISCLOSURE

In one aspect or embodiment, a method for site assessments of a catheter insertion site and/or dressing includes: scanning the catheter insertion site and/or dressing with an image capture device and/or sensor; selecting a patient baseline site location and skin tone; recording a baseline condition using a computing device; determining a site assessment rate using a computing device; prompting a clinician to make a site assessment of the catheter insertion site and/or dressing using a computing device; and recording site assessment information in an electronic medical record using a computing device.

The method may further include: scanning a proposed catheter insertion site prior to insertion of a catheter with the image capture device and/or sensor. The patient baseline site location and skin tone may be selected from a series of standardized images. The series of standardized images may be displayed by a computing device based on the scanning of the catheter insertion site and/or dressing with the image capture device and/or sensor. The site assessment rate may be determined by the computing device using at least one of: catheter type and location; current medication; patient status; patient physiology; patient mobility; and history of vascular access complications. The site assessment of the catheter site and/or dressing may include selecting an image from a series of standardized images.

The method may include identifying changes of the catheter insertion site and/or dressing over a predetermined period of time using a computing device. The changes of the catheter insertion site and/or dressing may include at least one of: movement of the catheter or dressing; changes in temperature of the catheter insertion site; changes in color of the catheter insertion site; changes in stiffness of the catheter insertion site; changes in density of the catheter insertion site; changes in reflectance and refraction of light of the catheter insertion site; and changes in three-dimensional profile of the catheter insertion site and/or dressing.

The method may include placing a reference marker on or adjacent to the catheter insertion site and/or dressing; and scanning the reference marker with the image capture device and/or sensor. The reference marker may include an information tag and a series of standardized colors. The method may include capturing an image of the catheter insertion site and/or dressing using the image capture device; and calibrating the image based on the reference marker using a computing device.

In a further aspect or embodiment, a computer program product for site assessments of a catheter insertion site and/or dressing, includes at least one non-transitory computer-readable medium including instructions that, when executed by a computing device, cause the computing device to: scan the catheter insertion site and/or dressing with an image capture device and/or sensor; record a baseline condition based on a selected patient baseline site location and skin tone; determine a site assessment rate; prompt a clinician to make a site assessment of the catheter insertion site and/or dressing; and record site assessment information in an electronic medical record.

The at least one non-transitory computer-readable medium may further include instructions that, when executed by a computing device, cause the computing device to: display a series of standardized images based on the scan of the catheter insertion site and/or dressing with the image capture device and/or sensor. The at least one non-transitory computer-readable medium may further include instructions that, when executed by a computing device, cause the computing device to: identify changes of the catheter insertion site and/or dressing over a predetermined period of time. The at least one non-transitory computer-readable medium may further include instructions that, when executed by a computing device, cause the computing device to: capture an image using the image capture device; and calibrate the image based on the reference marker using a computing device.

In a further aspect or embodiment, a system for site assessments of a catheter insertion site and/or dressing includes: one or more image capture devices configured to capture at least one of an optical, infrared, and thermal image of the insertion site and/or dressing; one or more sensors configured to determine at least one of density, reflectance of light, refraction of light, color calibration, and electrical impedance; and one or more computing devices in communication with the one or more image capture device and the one or more sensors, the computing device configured to record site assessment information from the one or more image capture devices and the one or more sensors.

The one or more computing devices may be configured to provide an automatic notification for a clinician to make a site assessment of the catheter insertion site and/or dressing. The one or more computing devices may be configured to identify changes of the catheter insertion site and/or dressing over a predetermined period of time based on the site assessment information from the one or more image captures devices and the one or more sensors. The system may further include a reference marker including an information tag and a series of standardized colors. The one or more computing devices may be configured to capture an image using the image capture device and calibrate the image based on the reference marker.

In a further aspect or embodiment, a computer program product for site assessments of a catheter insertion site and/or dressing, comprising at least one non-transitory computer-readable medium including instructions that, when executed by a computing device, cause the computing device to: acquire image align orientation; segment dressing and skin surface; normalize relative to calibration markers; convolve with optimal kernel; enhance images for display and analysis; determine a threshold and comparing a computed area with a threshold; and compute an index and comparing to risk index.

### BRIEF DESCRIPTION OF THE DRAWINGS

The above-mentioned and other features and advantages of this disclosure, and the manner of attaining them, will become more apparent and the disclosure itself will be better understood by reference to the following descriptions of aspects of the disclosure taken in conjunction with the accompanying drawings, wherein:

FIG. 1 is a perspective view of a system for site assessments of a catheter insertion site and/or dressing according to one aspect or embodiment of the present application.

FIG. 2 is a perspective view of a system for site assessments of a catheter insertion site and/or dressing according to a further aspect or embodiment of the present application.

FIG. 3 is a schematic view of a system for site assessments of a catheter insertion site and/or dressing according to a further aspect or embodiment of the present application.

FIGS. 4A-4D are perspective views of images analyzed over time by the system of FIG. 1 or FIG. 2.

FIG. 5 is a flow diagram of a method for site assessments of a catheter insertion site and/or dressing according to one aspect or embodiment of the present application.

FIG. 6A is a perspective view of a catheter insertion site and/or dressing according to one aspect or embodiment of the present application, showing a reference marker.

FIG. 6B is an enlarged view of the reference marker of FIG. 6A.

FIG. 7 is a front view of a catheter insertion site and/or dressing according to a further aspect or embodiment of the present application, showing a reference marker.

FIG. 8 is a flow diagram of a method for site assessments of a catheter insertion site and/or dressing according to a further aspect or embodiment of the present application.

FIG. 9 is a flow diagram of a method of data analysis for site assessments according to one aspect or embodiment of the present application.

FIG. 10 is a flow diagram of a method for site assessments of a catheter insertion site and/or dressing according to a further aspect or embodiment of the present application.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. However, it is to be understood that the disclosure may assume various alternative variations, except where expressly specified to the contrary. It is also to be understood that the specific devices illustrated in the attached drawings, and described in the following specification, are simply exemplary aspects of the disclosure. Hence, specific dimensions and other physical characteristics related to the aspects disclosed herein are not to be considered as limiting. All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. A computing device may also be a desktop computer or other form of non-mobile computer. In non-limiting embodiments, a computing device may include a GPU. In non-limiting embodiments, a computing device may be comprised of a plurality of circuits.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments, a message may refer to a network packet (e.g., a data packet and/or the like) that includes data. It will be appreciated that numerous other arrangements are possible.

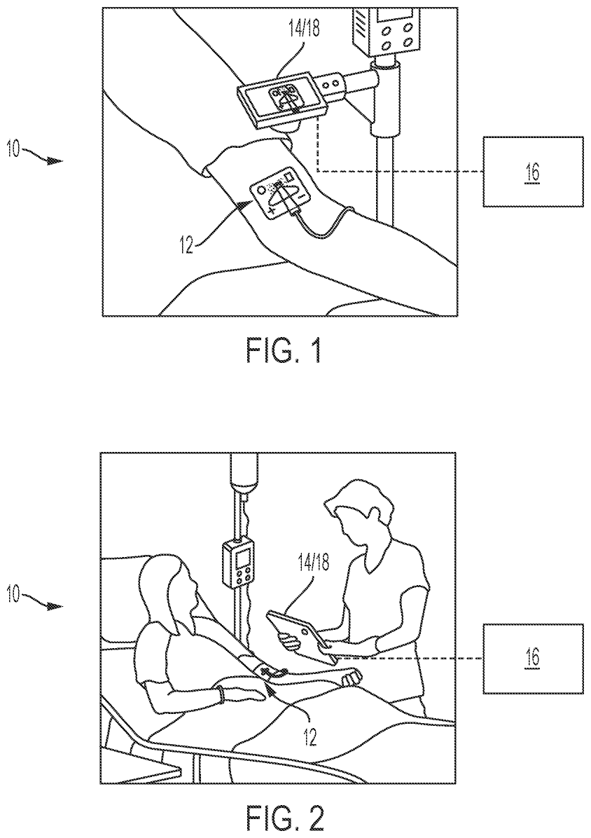

Referring to FIGS. 1-10, systems and methods for site assessments of a catheter insertion site and/or dressing are shown. The system may provide a clinician with pre-defined images of sites for the clinician to simply pick the image that is closest to the site on the patient they are observing. This will allow images to be tied to a standard scale, and enable analytics to tie scale to outcome. This will further enable earlier intervention if it is found that certain types of site issues lead to negative outcomes (e.g. peeling of dressing). For example, one or more of the categories currently described in the EMR (e.g. dressing status as “clean, dry, intact” vs “clean, intact, with new drainage”, etc., Phlebitis Grading Scale, infiltration) will have images to illustrate to the clinician what each subcategory grade is visualized as. To further enable this scale to standardize assessments, different images may be provided to illustrate different skin tones or placement on the body (e.g. placement on a patient's hand versus in the area of inflection on their elbow may look different).

Referring to FIGS. 1 and 2, a system 10 for site assessments of a catheter insertion site and/or dressing 12 may include one or more image capture devices 14, one or more sensors 16, and one or more computing devices 18 in communication with the one or more image capture device 14 and the one or more sensors 16. As detailed below, the computing device 18 is configured to provide or aid in providing an assessment of a catheter insertion site and/or dressing 12. The image capture device 14 and/or computing device 18 may be provided on a hand-held EMR tablet (FIG. 2) or may be provided on other equipment, such as an IV pole. A separate light source may be provided. The image capture device 14 and/or computing device 18 may be a hands-free voice-activated system.

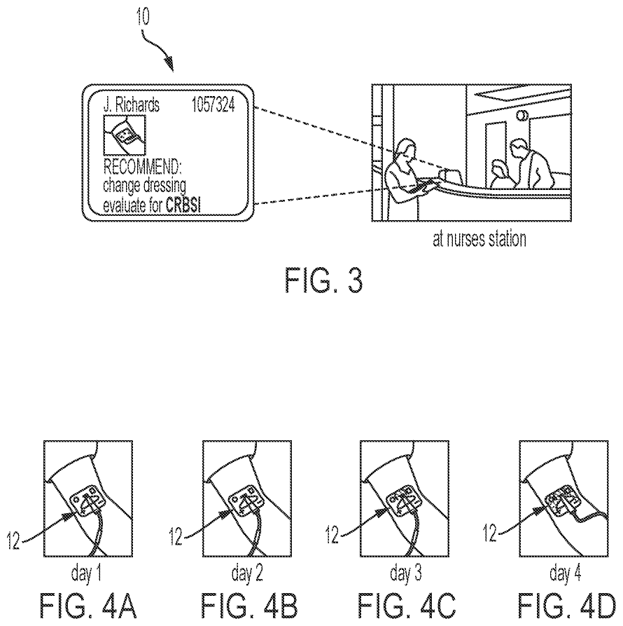

Referring to FIG. 3, the system 10 may provide automatic notification upon a first sign of infection and recommend an action. The system 10 may provide automatic notification to the computing device 18 after a predetermined period of time has elapsed since the last evaluation of the catheter insertion site and/or dressing 12.

Referring to FIGS. 4A-4D, images captured by the system 10 over a predetermined time period may be analyzed by the computing device 18 to detect changes, such as comparing site redness, swelling, irritation, temperature, and/or other signals at various time points. The images captured or recorded by the system 10 may a single still image, a set of images (e.g., a burst or series of consecutive images taken over a predetermined period of time), or a video or series of videos. As discussed below, the images may be color image, thermal images, depth images, or density topography images of the area or portions of the area.

The system 10 and method may utilize standardized images, such as pre-selected images to standardize reference scale, separate scales for dressing status, phlebitis grading, infiltration grading, etc. and/or subsets thereof, different sets for different skin tones and/or catheter placement sites, which may be auto-selected by a computer image analysis or based on clinician choice.

The system 10 and method may include the computer-implemented analysis of the following: image (optical, infrared, thermal, depth, etc.), property reflectance, tissue stiffness (e.g., tonometry); one parameter/property/image type or combination thereof; comparison of the site prior to and following catheter placement (undressed during dressing changes to assess skin directly) and/or following application of dressing and between dressing changes to assess dressing status); temporal analysis and/or variation within a single reading/image, e.g. assessment at two or more points within an image; and/or comparison of trends to historic data of similar patients to detect and/or anticipate complications.

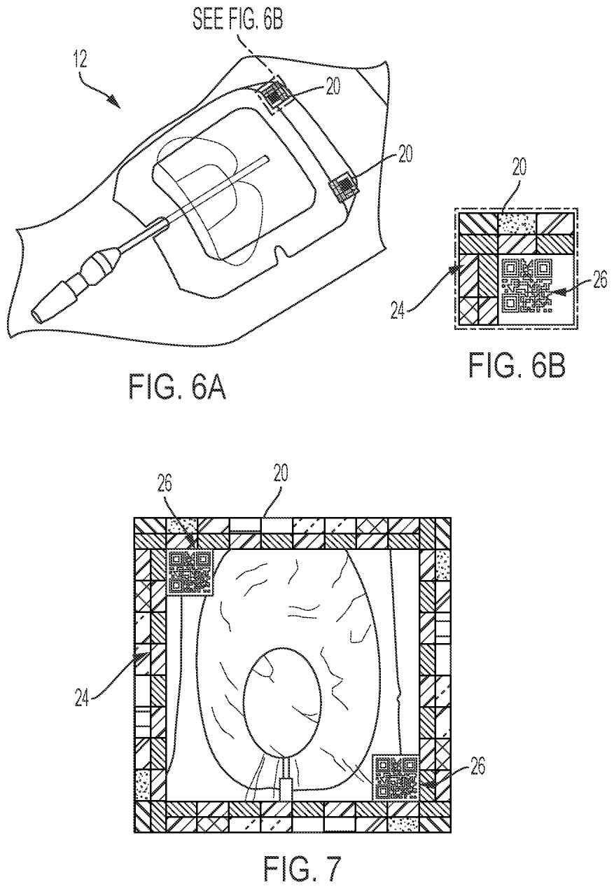

Referring to FIGS. 6A, 6B, and 7, the system 10 and method may utilize reference marker(s) 20 for the following: to calibrate and standardize images taken with different cameras; to correct for changes in lighting, ambient temperature, etc.; and/or potential reference to calibrate density meter or stiffness gauge. A separate reference marker 20 may be placed on body or the reference marker 20 may be provided on, or placed on the catheter and/or dressing. As shown in FIG. 6A, the reference marker(s) 20 is provided as a standalone sticker placed on the catheter and/or dressing. As shown in FIG. 7, the reference marker(s) 20 are formed integrally with the dressing. The reference marker(s) 20 may be multi-parametric and include several colors, textures, or physical features 24. The features 24 may be a series of standardized colors to allow for the standardization and calibration of images captured using the image capture device 14. The reference marker(s) include an information tag 26, such as a barcode, QR code, NFC tag, RFID tag, etc. The information tag 26 can be read or scanned by the system 10 and automatically bring up a patient record, enter information into a patient record, and/or facilitate the tracking, entry, and management of the patient records and information as discussed herein. The reference marker(s) 20 could further serve as a reference for surface compressibility of the skin and potentially have areas of defined compressibility that enables calibration of a tissue density measurement device. The reference marker(s) 20 may be incorporated into a full dressing, a full dressing overlay, a dressing frame, and/or a dressing sticker.

The system 10 may include the following: image capture (optical, thermal, depth, etc.); density/stiffness meter; capture of other parameters e.g. reflectance, refraction of light (to assess wetness); spectroscopic features which can be used for color calibration, or indicate infective or immune processes; stereoscopic imaging, radar, or other 3D depth techniques, to determine a three dimensional profile of the dressing, catheter insertion site, or area where the catheter is subdermal in order to, for example, detect inflammation, swelling or infiltration; electrical characteristics like impedance; and/or reactive dye that could be incorporated into dressing or applied to insertion sites.

In some aspects or embodiments, the system 10 will automatically show the clinician images similar to the patient's actual skin tone and location of the catheter based on initial imaging of the catheter insertion site and/or dressing 12 prior to catheter insertion. In other aspects or embodiments, the clinician may have to select which image set is most reflective of the patient's coloring during initial patient setup, or there may be multiple panels shown for the placement site assessment, and thereafter images similar in tone to the patient will be provided.

In one aspect or embodiment, the system 10 will assess the catheter insertion site and/or dressing 12 (with an optical, thermal, depth and/or reflectance based image and/or force based measurements to assess tissue stiffness). This will enable the computerized assessment of: whether the catheter is properly in place, has not been dislodged, whether the dressing is secure, is not peeling, dirty or otherwise compromised, and/or if the skin around the catheter insertion site is not discolored, warm or soft (via colorimetric analysis and/or thermal analysis at multiple points on the image. By asking the clinician to take images prior to and repeatedly following insertion, the computing device 18 will have the ability to observe, record, and/or evaluate site progression over time and compare it with outcomes from other patients to enable the computing device 18 to detect and predict occurrence of catheter-related complications (including infiltrations, extravasations, phlebitis, dislodgement) or local site infections.

Computerized assessment may include: “deep learning”, predictive analytics, data mining, or other pattern recognition as one of the methods of training an image classifier with a set of images where the status is known. The computerized assessment may include machine learning, image processing, artificial intelligence, and/or object detection.

Pattern recognition computations may further identify trends between images and/or within single images. For example, if the temperature profile of the catheter insertion site 12 is initially uniform, but then starts to demonstrate formation of a hot spot or cold spot, it might indicate that phlebitis or infiltration/extravasation, respectively, are starting to occur. A similar colorimetric analysis may also be performed to assess bruising.

In addition to imaging the catheter insertion site and/or dressing 12 itself, a more comprehensive image of the patient may also be used to assess status. For example, with catheters placed on or near the hand, imaging and image analysis of the fingers may serve to assess if blood flow to the fingers has been compromised (e.g. if a dressing is too tight or if infiltration/extravasation has led to restriction of blood flow).

To further standardize assessments, in some aspects or embodiments, the assessments of the catheter insertion site and/or dressing 12 may be performed dynamically based on patient specific parameters. For example: catheter type and location; infusate/current medications (e.g. vesicants and other irritant drugs may warrant more frequent assessment); patient status (e.g. patients with developmental venous anomalies (DVA) will require more frequent site assessments); patient physiology (age, sex, BMI, comorbidities, e.g. older patients may have more fragile veins or patients with a history of frequent intravenous injections); activities of daily living index/mobility patients who are more mobile may be more likely to accidentally dislodge a catheter during regular movement; history of vascular access complications (dislodgement, vesicants, vein depletion/fragility); and other factors identified based on trends of prior patients with similar parameters that are found to warrant extra monitoring.

The system 10 may include sensors and systems to calculate or measure the degree of patency of an IV line.

In one aspect or embodiment, a standardized assessment may be utilized with pictures and/or words that are representative of various states of the catheter insertion site and/or dressing 12. The images may be color coded based on patient skin type to give a more representative assessment. An image of the catheter insertion site and/or dressing 12 may be captured to determine and record if the site is clean, dry, intact, and/or to determine and record where the catheter has been placed and whether it has been placed properly. The system may include a dynamic recommendation of when site assessment should be performed based upon patient specific parameters which may include one or more of the following: catheter type; infusate (e.g. vesicants and other irritant drugs may warrant more frequent assessment); and patient status (e.g. DVA patients will require more frequent site assessments).

The system 10 may utilize various dressing assessment tools. In one aspect or embodiment, pre-defined images may be provided to standardize dressing assessment by a nurse. Different images for different skin tones may be provided. The computing device 18 can automatically determine what image set to provide based on an image of the patient to determine skin tone.

The use of standardized images may be utilized during the following scenarios: before catheter placement to determine skin tone and patient specific “normal”; following initial placement to assess quality of placement and site selection (e.g. near point of inflection, if line is kinked, if line is taped down well, if site was shaved, if dressing is intact/well adhered); during dressing changes to assess change when skin is directly able to be observed, e.g., skin coloration, wound dryness (versus wet because of fluid leakage or blood leakage, etc.); and between dressing changes, e.g., before line accesses or at defined time points or combination thereof. The time period may be defined based on patient specific parameters (e.g., age (toddlers more likely to have IV taken out due to mobility)), mobility, recent pump disconnects (e.g., may indicate patient went to recovery room), other patient parameters (hairiness, mental condition, e.g., psychosis or other psychological issues may make patient more likely to pull out IV, etc.), as well as what is being infused (e.g., vesicants or other irritant drugs may require more frequent assessments) to assess intactness of dressing and changes of dressing integrity and site, i.e., skin and area around catheter. During these scenarios, the following catheter issues may be evaluated: line dislodgment or migration (catheter may have indicator to demonstrate skin insertion level and assessment of the distance between that and skin may indicate dislodgement or migration, or visual evidence of catheter tip or portion of catheter being out of vein); line kinks; dressing intactness; site cleanliness (shaved/hair removed); and site (dry/not wet, no blood, no fluid present, patient not sweating).

The image capture device 14 and/or sensor 16 are utilized to record site characteristics and may include both images and/or other measurements, as discussed above. The image may have a color spot to enable auto-adjustment of color in different lighting. The image may also include a thermal image. The image may include reflectance of light (to assess wetness: blood, sweat, fluid leak). In addition to the image, the system 10 may also take measurement of tissue stiffness or compressibility, e.g. via pressure, tension meter, force gauge, etc. via the sensor 16. An energy source, such as a light source, pressure source, tension source, heat source, acoustic/vibration source that is used to stimulate the test area, and a corresponding sensor that detects transmitted or reflected energy to assess a property of the test area that indicates its status may be utilized. Any of these energy sources can be modulated to test multiple sample points or characteristics.

Various computer-implemented methods of site assessment may be utilized including, for example, the following: a method to compare baseline parameters of site prior to placement with variation over time to identify changes and/or trends indicative of or predicative of negative outcomes; a method which utilizes colorimetric analysis; a method which utilizes thermal analysis; a method which uses other property analysis (e.g. reflectance or other surface property assessment); a method assessing site during dressing changes (i.e., bare skin) over time or site through/under dressing; and/or a method that may draw conclusions about changes in skin color or shape or tissue compressibility (e.g., look at shadows created in image correcting for angle and other artifacts to assess if swelling is present) and other changes potentially indicative of swelling (e.g. blanching of skin, differences in light reflection or refraction).

The system 10 may present a clinician with a series of images captured over a predetermined period of time, such as every 2 hours or every 4 hours, with the images being corrected for orientation, color, lighting, angle, shadows, and other image characteristics to enable comparison of how elements of a site, such as redness or swelling, are progressing over time. These elements may be highlighted within the images presented to the clinician and may include a length scale or other type of scale. The images presented to a clinician may be enhanced with information or other indicators obtained by computer-aided image analysis of the image or previously captured images.

The system 10 may be configured to detect fluid buildup under skin, fluid buildup under dressing (blood, pus, wetness, oozing wound, etc.) and determine differences in shine, temperature, color (e.g. red blood), or combination thereof.

The system 10 may evaluate finger coloring, for sites close to fingers/on hand (or even further up arm), to see if circulation is compromised, which is a sign of infiltration/extravasation.

The system 10 may determine changes in temperature across skin, which may indicate phlebitis/infection for increases in temperature at the site or infiltration/extravasation for decreases in temperature.

The system 10 may determine if the dressing is too tight based on blanching of skin or other indications of improper dressing.

Images/readings taken from the catheter insertion site and/or dressing 12 may be used to document the site directly without computerized analysis in one aspect or embodiment. This would allow clinicians to evaluate site progression more directly without having to rely on memory.

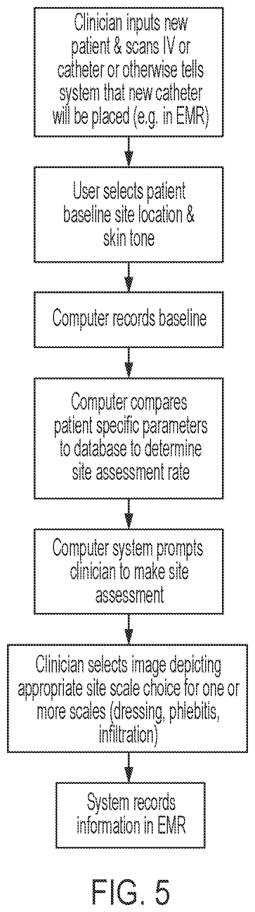

Referring to FIG. 5, in one aspect or embodiment, a method for site assessments of the catheter insertion site and/or dressing 12 includes: scanning the catheter insertion site and/or dressing 12 with the image capture device 14 and/or sensor 16; selecting a patient baseline site location and skin tone; recording a baseline condition using the computing device 18; determining a site assessment rate using the computing device 18; prompting a clinician to make a site assessment of the catheter insertion site and/or dressing 12 using the computing device 18; and recording site assessment information in an electronic medical record using the computing device 18.

The system 10 and method may include scanning a proposed catheter insertion site 12 prior to insertion of a catheter with the image capture device 14 and/or sensor 16. The patient baseline site location and skin tone may be selected from a series of standardized images as discussed above. The series of standardized images may be displayed by the computing device 18 based on the scanning of the catheter insertion site and/or dressing 12 with the image capture device 14 and/or sensor 16. As discussed above, the method may include identifying changes of the catheter insertion site and/or dressing 12 over a predetermined period of time using the computing device 18. The method may include placing the reference marker 20 on or adjacent to the catheter insertion site and/or dressing 12. In one aspect or embodiment, adjacent to the catheter insertion site and/or dressing 12 may refer to the reference marker 20 on or within a 12 inch radius from a center of the catheter insertion site and/or dressing 12. In one aspect or embodiment, adjacent to the catheter insertion site and/or dressing 12 may refer to the reference marker 20 is on or within a 6 inch radius from a center of the catheter insertion site and/or dressing 12. The method may include scanning the reference marker 20 with the image capture device 14 and/or sensor 16. As discussed above, the method may include capturing an image of the catheter insertion site and/or dressing 12 using the image capture device 14; and calibrating the image based on the reference marker 20 using a computing device 18.

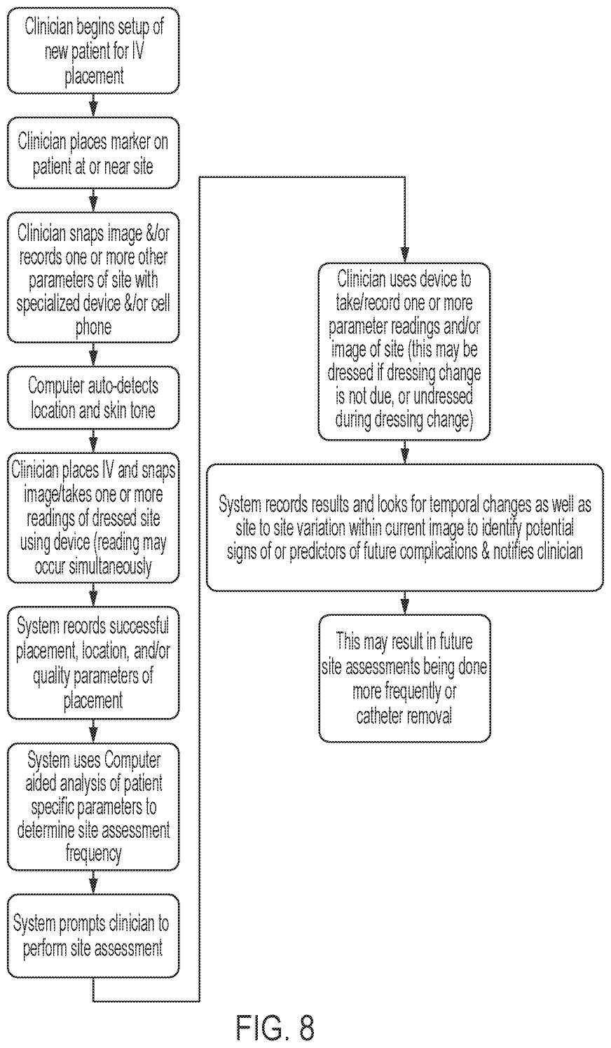

Referring to FIG. 8, in one aspect or embodiment, a method for site assessments of the catheter insertion site and/or dressing 12 includes: placing the reference marker 20 on a patient at or near the catheter insertion site 12; capturing an image or parameter of the catheter insertion site 12 with the image capture device 14 or sensor 16; auto-detecting a location and skin tone of the catheter insertion site 12 using the computing device 18; placing a catheter and capturing an image of the catheter insertion site and/or dressing 12; recording a placement, location, and/or parameters of the catheter insertion site and/or dressing 12; determining site assessment rate or frequency based on patient specific parameters using the computing device 18; and prompting a clinician to perform a site assessment using the computing device 18.

### Data Analysis

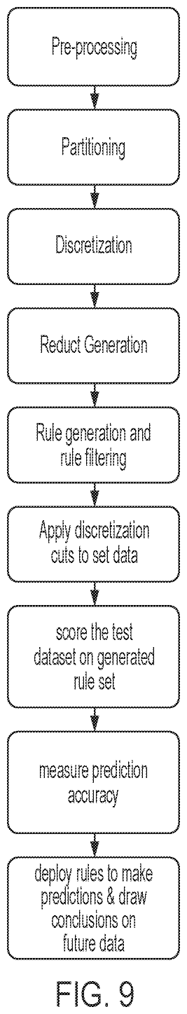

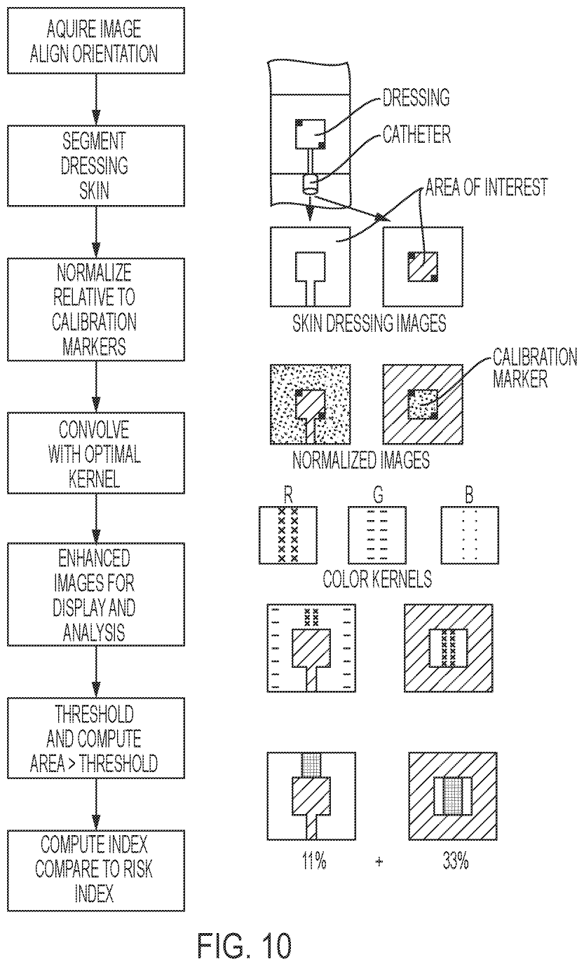

Referring to FIGS. 9 and 10, potential image processing techniques for application in site assessments includes: spatial domain processing, frequency domain processing, and non-Abelian group operations; neural networks and deep learning, e.g. gradient descent algorithms, backpropagation, matrix-based algorithms; morphological image processing, blob analysis, connectivity analysis and set theory evaluating shape and size, etc., including image processing operations such as dilation, erosion, opening, closing, etc.; nth nearest neighbor analysis, classification, clustering, regression, association rules, outer detection, sequential patterns, anomaly or outlier detection, prediction/induction rule, summarization, sequential patterns, decision tree learning, tracking patterns, statistical techniques (probabilistic models, inference), visualization, neural networks, data warehousing, association rule learning, long-term memory processing, and prediction; enhancement, restoration, encoding, transformation, and compression; rough sets; vector machines including primal form, dual form, soft margin; and non-linear classification including regression, implementation, and kernel trick. The image processing techniques may include: artificial intelligence; machine learning; time domain processing; video processing and analytics; feature extraction and feature matching; image registration techniques; image sticking; convolution; morphological techniques; image transformation; orientation and illumination correction; normalization; warping and de-warping; calibration; long short-term memory (LSTM) and supervised, unsupervised and reinforcement learning; edge detection; occlusion detection; image restricting and reconstruction; cropping, contrast, and brightness correction; masking, color conversion, grayscale, and image comparison and analysis; object detection and classification; segmentation and augmentation; depth mapping and analysis; 3D imaging; and/or florescent imaging techniques.

As shown in FIG. 9, data analysis according to one aspect or embodiment may include pre-processing; partitioning; discretization; reduct generation; rule generation and rule filtering; applying discretization cuts to set data; scoring the test dataset on generated rule set; measuring prediction accuracy; and deploying rules to make predictions and draw conclusions on future data.

Data processing and analysis may be conducted on images, subsets thereof, or other data collected by the device alone or in combination with each other.

Image processing may include splitting an image into relevant spectrum subsets, e.g. <525, 535-550, 550-600, 600-660, >660 (Red), optionally converting to grayscale, and looking for patterns in image density over time, e.g. migrating density to higher or lower spectrum (to identify increases in blue (bruising) or red (infection). This could be coupled with thermal imaging and pattern recognition considering temperature differences between the site of entry and one or more points or radiuses more distal from the point of entry and considering trends of thermal changes wherein the temperature difference in the thermal image demonstrate an increasing hot spot near the site of entry with heat that builds centrally and radiates outward may indicate phlebitis (or a spot of cooling around the point of entry to indicate infiltration/extravasation).

Referring to FIG. 10, a method of providing a skin inflammation index algorithm may include: acquiring image align orientation; segmenting the dressing and skin surface; normalizing relative to calibration markers; convolving with optimal kernel; enhancing images for display and analysis; determining a threshold and comparing a computed area with a threshold; and computing an index and comparing to risk index.

The system 10 may perform one or more processes described herein. The system may perform these processes based on a processor executing software instructions stored by a computer-readable medium, such as memory and/or storage component. A computer-readable medium may include any non-transitory memory device. A memory device includes memory space located inside of a single physical storage device or memory space spread across multiple physical storage devices. Software instructions may be read into memory and/or storage from another computer-readable medium or from another device via a communication interface. When executed, software instructions stored in memory and/or storage component may cause a processor to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

While this disclosure has been described as having exemplary designs, the present disclosure can be further modified within the spirit and scope of this disclosure. This application is therefore intended to cover any variations, uses, or adaptations of the disclosure using its general principles. To the extent possible, one or more features of any embodiment or aspect can be combined with one or more features of any other embodiment or aspect. Further, this application is intended to cover such departures from the present disclosure as come within known or customary practice in the art to which this disclosure pertains and which fall within the limits of the appended claims.

1. A method for site assessments of a catheter insertion site and/or dressing comprising:
scanning the catheter insertion site and/or dressing with an image capture device and/or sensor;
selecting a patient baseline site location and skin tone;
recording a baseline condition using a computing device;
determining a site assessment rate using a computing device;
prompting a clinician to make a site assessment of the catheter insertion site and/or dressing using a computing device; and
recording site assessment information in an electronic medical record using a computing device.

2. The method of claim 1, further comprising:
scanning a proposed catheter insertion site prior to insertion of a catheter with the image capture device and/or sensor.

3. The method of claim 1, wherein the patient baseline site location and skin tone is selected from a series of standardized images.

4. The method of claim 3, wherein the series of standardized images are displayed by a computing device based on the scanning of the catheter insertion site and/or dressing with the image capture device and/or sensor.

5. The method of claim 1, wherein the site assessment rate is determined by the computing device using at least one of: catheter type and location; current medication; patient status; patient physiology; patient mobility; and history of vascular access complications.

6. The method of claim 1, wherein the site assessment of the catheter site and/or dressing comprises selecting an image from a series of standardized images.

7. The method of claim 1, further comprising:
identifying changes of the catheter insertion site and/or dressing over a predetermined period of time using a computing device.

8. The method of claim 1, wherein the changes of the catheter insertion site and/or dressing comprises at least one of: movement of the catheter or dressing; changes in temperature of the catheter insertion site; changes in color of the catheter insertion site; changes in stiffness of the catheter insertion site; changes in density of the catheter insertion site; changes in reflectance and refraction of light of the catheter insertion site; and changes in three-dimensional profile of the catheter insertion site and/or dressing.

9. The method of claim 1, further comprising:
placing a reference marker on or adjacent to the catheter insertion site and/or dressing; and
scanning the reference marker with the image capture device and/or sensor.

10. The method of claim 9, wherein the reference marker comprises an information tag and a series of standardized colors.

11. The method of claim 9, further comprising:
capturing an image of the catheter insertion site and/or dressing using the image capture device; and
calibrating the image based on the reference marker using a computing device.

12. A computer program product for site assessments of a catheter insertion site and/or dressing, comprising at least one non-transitory computer-readable medium including instructions that, when executed by a computing device, cause the computing device to:
scan the catheter insertion site and/or dressing with an image capture device and/or sensor;
record a baseline condition based on a selected patient baseline site location and skin tone;
determine a site assessment rate;
prompt a clinician to make a site assessment of the catheter insertion site and/or dressing; and
record site assessment information in an electronic medical record.

13. The computer program product of claim 12, wherein the at least one non-transitory computer-readable medium further includes instructions that, when executed by a computing device, cause the computing device to:
display a series of standardized images based on the scan of the catheter insertion side and/or dressing with the image capture device and/or sensor.

14. The computer program product of claim 12, wherein the at least one non-transitory computer-readable medium further includes instructions that, when executed by a computing device, cause the computing device to:
identify changes of the catheter insertion site and/or dressing over a predetermined period of time.

15. The computer program product of claim 12, wherein the at least one non-transitory computer-readable medium further includes instructions that, when executed by a computing device, cause the computing device to:
capture an image using the image capture device; and
calibrate the image based on the reference marker using a computing device.

16. A system for site assessments of a catheter insertion site and/or dressing comprising:
one or more image capture devices configured to a capture at least one of an optical, infrared, and thermal image of the insertion site and/or dressing;
one or more sensors configured to determine at least one of density, reflectance of light, refraction of light, color calibration, and electrical impedance; and
one or more computing devices in communication with the one or more image capture device and the one or more sensors, the computing device configured to record site assessment information from the one or more image capture devices and the one or more sensors.

17. The system of claim 16, wherein the one or more computing devices is configured to provide an automatic notification for a clinician to make a site assessment of the catheter insertion site and/or dressing.

18. The system of claim 16, wherein the one or more computing devices is configured to identify changes of the catheter insertion site and/or dressing over a predetermined period of time based on the site assessment information from the one or more image captures devices and the one or more sensors.

19. The system of claim 16, further comprising a reference marker comprising an information tag and a serious of standardized colors.

20. The system of claim 19, wherein the one or more computing devices is configured to capture an image using the image capture device and calibrate the image based on the reference marker.
